(19) United States
(12) Patent Application Publication
(10) Pub. No.: US 2022/0109512 A1
(43) Pub. Date: Apr. 7, 2022

(54) Secured Intrabody Networks and Interfaces for the Internet of Things and Multiple Uses of Ultrasound Wideband

(71) Applicant: BIONET SONAR, Newton, MA (US)

(72) Inventors: Tommaso Melodia, Newton, MA (US); Jorge Jimenez, Atlanta, GA (US); Emrecan Demirors, Boston, MA (US)

(21) Appl. No.: 17/428,338

(22) PCT Filed: Feb. 5, 2020
(86) PCT No.: PCT/US2020/016799

§ 371 Date: Aug. 4, 2021

Related U.S. Application Data
(60) Provisional application No. 62/802,023, filed on Feb. 6, 2019.
(60) Provisional application No. 62/802,276, filed on Feb. 7, 2019.

(51) Int. Cl. H04B 13/00; H04B 11/00; G06F 21/62; G06Q 20/38

(52) U.S. Cl. CPC H04B 13/005; G06Q 20/3821; G06F 21/6245; H04B 11/00

(57) ABSTRACT

Systems, methods, and computer-readable media are disclosed for secured intrabody networks and interfaces for IoT and ultrasound wideband. Example devices may include a wearable device in contact with a surface of a human body, the wearable device including an ultrasonic wave generator configured to transmit ultrasonic waves, and an external receiver configured to receive the ultrasonic waves and determine data encoded in the ultrasonic waves. The ultrasonic waves may encode a single bit of data in multiple pulses through a pseudorandom time hopping scheme.

### CROSS-REFERENCE TO RELATED APPLICATIONS

This application claims the benefit of both U.S. Provisional Application Ser. No. 62/802,023, filed Feb. 6, 2019, and U.S. Provisional Application Ser. No. 62/802,276, filed Feb. 7, 2019, both of which are hereby incorporated by reference in their entireties.

### BACKGROUND

In recent years, there has been a lot of interest on intrabody networks and the Internet of Things (IoT) in general. Intrabody networks could be used for miniaturized implant and or wearables for multiple uses including medical treatment/monitoring/diagnosis, identification, secured communications, and privacy. The internet of medical things is the basis of the modern interconnected world, through the interaction of different elements interconnected as nodes and actuators in a network. However, communicating data using intrabody networks may be difficult.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. In the drawings, the left-most digit(s) of a reference numeral may identify the drawing in which the reference numeral first appears. The use of the same reference numerals indicates similar, but not necessarily the same or identical components. However, different reference numerals may be used to identify similar components as well. Various embodiments may utilize elements or components other than those illustrated in the drawings, and some elements and/or components may not be present in various embodiments. The use of singular terminology to describe a component or element may, depending on the context, encompass a plural number of such components or elements and vice versa. For example, the term “a clinical trial identifier” can refer to one or more identifiers and clinical trials.

FIG. 1 is a schematic diagram of example continuous and pulsed wave ultrasound in accordance with one or more example embodiments of the disclosure.

FIG. 2 is a schematic diagram of components of ultrasonic based communication devices in accordance with one or more example embodiments of the disclosure.

FIG. 3 is example schematic process flow diagram for a payment or communication scheme in accordance with one or more example embodiments of the disclosure.

FIG. 4 is an example implementation of ultrasonic based communication scheme for payment in terminals in accordance with one or more example embodiments of the disclosure.

FIG. 5 is an example process flow diagram for payments using ultrasonic based communication schemes in accordance with one or more example embodiments of the disclosure.

FIG. 6 is a schematic illustration of example computer architecture of a wearable device in accordance with one or more example embodiments of the disclosure.

### DETAILED DESCRIPTION

### Overview

Embodiments described herein provide devices, systems, communication protocols, wireless links, and methods for creating wireless networks of devices inside and outside of a human or animal body, thereby allowing for improved secure data transmission. The systems described herein use ultrasound technology to establish communication through human tissue by transmitting ultrasonic waveforms modulated in an optimized manner so as to be able to carry information or data reliably, covertly, privately, and in an energy efficient fashion through human or animal tissue. The physical implementation of such a communication technology may be through implanted hardware or in an embodiment using wearables configured to communicate with such implants or with devices external to the body. In an embodiment, a wearable transmitter (e.g., watch or watch band, bracelet, necklace, patch, clothing, footwear, etc.) may be configured to use a piezoelectric element or other ultrasound generator to send mechanical waves through the human body carrying data within the ultrasonic waveforms, which may then be acquired by another piezoelectric or ultrasonic element at a receiver in a different location.

In an embodiment, pulsed ultrasonic waves are used to transmit bits of encoded data, where some or all, or each, bit is encoded in one or more, or multiple, pulses using a pseudo-random time hopping scheme. At the receiver, packet synchronization and time-hopping synchronization may be performed to decode the received signal. In an embodiment, the receiver of the ultrasonic waves may be a payment station, intelligent credit payment card, cell phone, mobile device, or other electronic device configured to facilitate secured payment, encrypted, and/or private communication.

This disclosure relates to, among other things, systems, methods, computer-readable media, techniques, and methodologies for secured intrabody networks and interfaces for IoT and ultrasound wideband. Some embodiments may be used to allow the human body to act as a network substrate itself, a medium for communication, an actuator, and/or a node in the internet of things. The use of ultrasound based communication and energy transmission allows communication and energy transmission through tissue and other bodily elements so that implants and/or wearables on the body may be used as nodes in an intrabody network, and with appropriate interfaces these elements or network can connect to an external internet of things. Unlike conventional technology, embodiments may overcome limitations associated with radio frequency (RF) based communication technologies.

Classical wireless communications are unsuitable for intrabody data transfer because wireless communications are today largely based on RF electromagnetic waves, and specifically microwaves, which are the physical basis of commercial wireless technologies like Wi-Fi, Bluetooth, LTE, and others. RF wireless has limitations in a number of applications. For example, RF wireless signals are heavily absorbed when propagating in proximity or inside biological tissues. Moreover, RF wireless signals can be jammed or intercepted, and may be unable to operate in the presence of radiological attacks. As a consequence, (i) RF based transmission may not be confined in biological tissues; (ii) absorption limits depth of signal penetration for data or energy transmission; (iii) because they propagate outside of the body, signals transmitted with RF can be detected, recorded, and potentially eavesdropped.

This disclosure relates to systems, methods, and devices to establish communication through human tissues based on transmitting ultrasonic waveforms—sound at inaudible frequencies modulated in an optimized manner, so as to be able to carry information reliably, covertly, privately, and in an energy efficient fashion through tissue. Ultrasonic wideband technology, as described in embodiments here, may be used for the creation of software defined wireless links and networks connecting two or more implantable or wearable devices—where the signal propagates through biological tissues. As a result, the data is confined inside the body unless changes are made to receiver so that they may also advance in air. Acoustic waves, typically generated through piezoelectric materials, may propagate better than their RF counterpart in media composed mainly of water. Piezoelectrically generated acoustic waves may be used for applications, among others, in underwater communications (typically at frequencies between 0 and 100 kHz), in indoor localization in sensor networks, and in ultrasonic medical imaging. While communication at low frequencies requires sizable transducers, innovations in piezoelectric materials and fabrication methods, primarily driven by the need for resolution in medical imaging, have made miniaturized transducers, at the micro, and even nano scales a reality. Moreover, the medical experience of the last decades has demonstrated that ultrasounds are fundamentally safe, as long as acoustic power dissipation in tissues is limited to predefined safety levels. It is also known that ultrasonic heat dissipation in tissues is minimal compared to RF waves.

Embodiments of the disclosure include a number of technical features that may be implemented to provide intrabody networks for the communication of data. Example technical features include generation of ultrasonic waves that may be pulsed and may carry encoded data. Such waveforms may be transmitted through human or animal tissue. Certain embodiments may use ultrasonic waves to authenticate payment transactions, user identity, user access to secured spaces, and so forth. The above examples of technical features and/or technical effects of example embodiments of the disclosure are merely illustrative and not exhaustive.

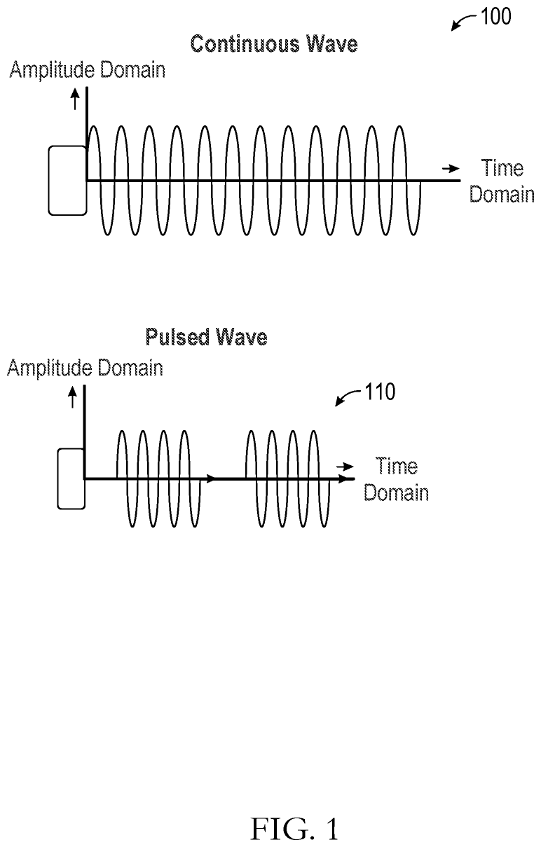

Referring to FIG. 1, an example of a continuous wave 100 and a pulsed wave 110 is presented. The continuous wave 100 may be generated using an ultrasound probe and may have a continuous waveform generated over time. The continuous wave 100 may have any suitable amplitude. In contrast, the pulsed wave 110 may be generated using an ultrasound prove and may be generated in bursts or pulses over time, such that discrete waveforms are generated over a time period. Unlike the continuous wave 100, the pulsed wave 110 may not be a single continuous waveform, and instead may include a number of pulsed waveforms separated by gaps in which no waveform is produced.

Embodiments of the disclosure may use ultrasonic wide band to establish communication through human tissues based on transmitting ultrasonic waveforms modulated in an optimized manner so as to be able to carry information reliably, covertly, privately, and in an energy efficient fashion through tissue.

The proposed ultrasonic transmission and multiple access technique, which may be referred to as Ultrasonic WideBand (UsWB), may be used for: 1) low-complexity and reliable communications in ultrasonic channels against the effect of multipath reflections within the human body; 2) limiting the thermal effect of communications, which is detrimental to human health; 3) enabling distributed medium access control and rate adaptation to combat the effect of interference from co-located and simultaneously transmitting devices. Ultrasonic wideband, as described herein, may be configured to provide physical-layer functionalities and medium access control arbitration and adaptation to enable multiple concurrent co-located transmissions with minimal coordination.

Data in the ultrasonic signal may be encoded in the time (frequency/period) domain, the amplitude domain, and/or in the phase and or amplitude shift of the wave. Ultrasonic signals for data transmission can be both continuous and/or pulsed as shown in FIG. 1. In continuous signals data, specifically, each byte of data can encoded in different sections of the time domain. In such domain the byte of data can be encoded in the frequency itself of in the frequency shift. The frequency may be shifted from a single reference point or from multiple points across the time domain. In some embodiments, the byte of data is encoded by bits in adjacent section of the signal or in sections which are separated by a known period. In some embodiments, each byte of data may be encoded by bits presented at the same time point, in the amplitude domain. In such embodiments, multiple waves with specific amplitudes may be used in order to encode all the bits required to construct the data byte. Both encoding in the time and amplitude domain may be accomplished by using a single or multiple ultrasonic waves generated by a single or multiple emitters. In an embodiment, pulsed ultrasonic waves are used to transmit the data.

In an embodiment using pulsed ultrasound to transmit data, the ultrasonic wave is truncated in time to create different wave packages or pulses. Each package contain a section of wave and a single pulse or multiple pulses may be used to encode a bite of data. In an embodiment each single byte of data is encoded in multiple pulses which carry the bits that construct the specific bite. Therefore, embodiments may use ultrasonic wideband to transmit data through soft tissues, bone, fluid, other solids or even air by encoding bites of data using multiple and discreet ultrasound pulses to transport the bits that conform the corresponding byte.

In an embodiment, data is transmitted by using very short ultrasonic pulses following an adaptive time-hopping pattern together with a superimposed adaptive spreading code. Baseband pulsed transmissions may be used for high data rate, low-power communications, and low-cost transceivers. Pulsed transmission delay resolution properties may be suited for propagation in the human body, where inhomogeneity in terms of density and propagation speed, as well as the pervasive presence of very small organs and particles, cause dense multipath and scattering. When replicas of pulses reflected or scattered are received with a differential delay at least equal to the pulse width, they may not overlap in time with the original pulse. Therefore, for pulse durations in the order of hundreds of nanoseconds, pulse overlaps in time are reduced, and multiple propagation paths can be efficiently resolved and combined at the receiver to reduce a bit error rate.

In an embodiment, the low duty cycle of pulsed transmissions is used to reduce the impact of thermal and mechanical effects, which can be detrimental for human health. In these embodiments, the transmission wave is designed such that the relationship between the period between neighboring pulses and the amplitude and/or energy contained within such a pulse is modulated in order to ensure that local temperature in the tissue that comprises the channel that transports the ultrasonic wave does not sustain an increase in temperature of more than about one to about five degrees Celsius, such as about three degrees Celsius. Pulse period and energy of the transmitted package can be designed into the specific transmission wave to ensure a known transfer of energy or thermal exchange, or an automated algorithm may be programmed into the emitter to automatically modulate the ultrasonic wave architecture to control energy transfer and heat exchange. In some embodiments, a temperature determined using a single or multiple temperature sensors at the transmitter or in the tissue through the waves path can be used in a closed loop feedback to modulate the ultrasonic wave architecture in order to ensure acceptable changes in temperature within the tissue.

In some embodiments, pulsed ultrasonic waves also allow for large instantaneous bandwidth and fine time resolution for accurate position estimation and network synchronization. In some instances, interference mitigation techniques may be used to enable MAC protocols that do not require mutual temporal exclusion between different transmitters. These interference mitigation techniques also may be used to reduce the effect of reflections in bone-tissue interfaces or other changes in medium within the human body, and thus improve reliability of the received data.

In some embodiments, further reduction in interference from other transmitters or reflections may be accomplished by using an adaptive channel code, by dynamically regulating the coding rate to adapt to channel conditions and interference level. The adaptive channel code may be a pseudo-orthogonal spreading code that is used because of the multiple access performance, limited computational complexity, and inherent resilience to multipath. Embodiments of modulation schemes for the adaptive channel scheme include PPM-BPSK-spreads and PPM-PPM-spreads. In PPM-BPSK-spread, the information bit is spread using BPSK modulated chips, and by combining with time-hopping. In PPM-PPM-spread, the information bit is spread using PPM-modulated chips.

In an embodiment, bits encoded in different pulses use adaptive time hopping scheme as structure to construct bytes of information. Each user transmits based on a pseudorandom time-hopping sequence, a sequence generated by seeding a random number generator with the unique identifier. The train of pulses is modulated based on pulse position modulation. A longer time frame reduces the interference generated to the other users. At the receiver, packet synchronization and time-hopping synchronization may be performed to properly decode the received signal.

In some embodiments, packet synchronization may include finding the correct time instant corresponding to the start of an incoming packet at the receiver. In general, this can be achieved through an energy-collection approach. During the packet synchronization, the transmitter sends an a priori known sequence or preamble. After correlating the received signal and the expected signal, the receiver identifies the starting point of the packet as the time instant where the correlation is maximized. The second step includes finding the time-hopping sequence to hop position by position and correlate the received pulses. This can be achieved by seeding the random generator with the same seed used by the transmitter, and therefore generating the same pseudorandom time-hopping sequence. Once both synchronization processes have been accomplished, the receiver can decode the received signal by listening in the bits of interest and by correlating the received pulse according to the modulation scheme in use.

In some embodiments, medium access control and rate adaptation strategies designed to find optimal operating points along efficiency-reliability tradeoffs may be used. In some embodiments of medium access control, a rate-adaptation algorithm selects a pair of code and frame lengths, based on the current level of interference and channel quality measured at the receiver and on the level of interference generated by the transmitter to the other ongoing communications to improve reliability and efficiency. In some embodiments, the receiver estimates interference, and also calculates frame and spreading code lengths that maximize the system performance through the rate adaptation algorithm. System performance maximization includes variables associated with reliability of the communication scheme and energy efficiency of the system. Energy efficiency considers both energy per bit and average energy emitted per second.

Medium access control coordination may be achieved by exchanging information on logical control channels, while data packets are transmitted over logical data channels. In these coordination schemes, prior to transmitting a packet, a dedicated channel may first be reserved. The connection is opened through the common control channel, which can be implemented through a unique sequence and a spreading code known and shared by all network devices. Request-to-Transmit packets and Clear-to-Transmit control packets are sent under low energy and low interference conditions prior to establishing a dedicated channel. Using data from this initial packet exchange the receiver computes the optimal frame and spreading code lengths to maximize efficiency and reliability of the dedicated channel where the bulk of the information will be transmitted.

The strategies above may be used to create an advanced ultrasonic wideband waveform for efficient data transmission using ultrasonic waves within the body. This waveform may be adapted by changing transmission frequency and other variables in order to improve transmission in a specific part of the human body. Transmitters and/or receivers that transfer data using ultrasonic wideband technology as described herein may be placed in any location on the surface of the human body or implanted within (e.g., torso, extremities, head, etc.).

For wearable applications (e.g., devices in contact with the surface of the body and/or skin, etc.) transmitters or receivers may be in contact with the human arm, wrist, hand, or other body part. In an embodiment, an advanced waveform is specifically created to transmit data through the human arm, hand, wrist, or other body part to minimize error rate and energy consumption. Some embodiments may use custom waveforms to transmit though such sections of the body using frequencies between 100 and 200 KHz. In this embodiment, the waveform may be of a pseudo-random encoded sequence of carrier-less pulses generated digitally with a square wave that is filtered by the transducer resonant frequency. In a different embodiment, the same waveform can be generated digitally and then be converted to the analog domain with a digital to analog converter. The waveform can be received by receiver. An embodiment for a non-coherent receiver filter may adaptively detect the presence of pulses by means of a pilot pulse that is used to estimate the average power of the received signal. The waveform uses adaptive thresholding without using any phase information (non-coherent). In a different embodiment, the receiver is also able to leverage phase information to either improve the reliability or the data rate of the link.

In the embodiments presented above the transmitter and receiver may be presented at separate entities or hardware, but those skilled in the art understand that in bi-directional or multidirectional communication uses, both the transmitter and receiver may send or receive data. In some embodiments, the transmitter and/or receiver can send or receive data using the same piezoelectric or ultrasonic hardware. In such embodiments, discrete cycles are created to separate transmitting and receiving phases using a time clock or digital synchronization elements to coordinate receiver and transmitter functions between the communication nodes. In some embodiments, bi-directional communication algorithms may be simplified by hardware that uses multiple piezo-electric or ultrasonic emitting/receiving hardware in each communication node, so that one piezo or element may be dedicated to transmitting and another to receiving. In such applications, anti-interference schemes can be fundamental. In some embodiments, multiple piezo or ultrasound emitting elements/antennas in a single node may be used not only to divide the send/receive channels but also to enhance communication speed, reliability or efficiency using MIMO, beam forming or other multi input/output systems.

Embodiments may include ultrasonic wideband technology that is implemented in a miniaturized, integrated, implantable/wearable digital/analogue hardware, that may be the core elements of each communication node in the network. Core properties of ultrasonic wideband hardware platform may include: (i) high-rate and secure data transmission through ultrasound; (ii) first miniaturized ultrasonic transceiver available; (iii) power consumption orders of magnitude (at least two consisting of a receiver and a transmitter chain with significant signal) lower than RF; (iv) recharging of platform battery using ultrasound.

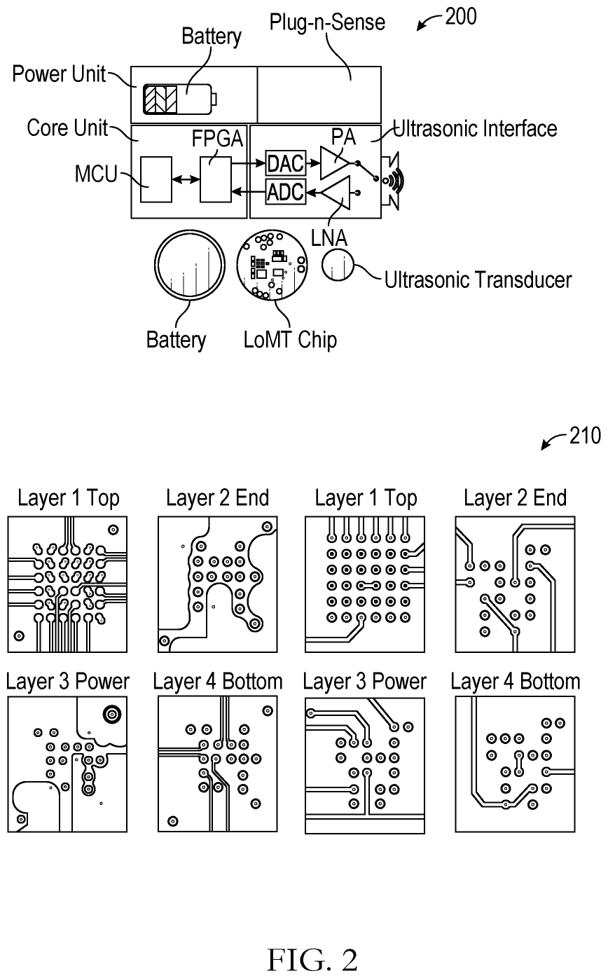

Referring to FIG. 2, in an example hardware embodiment, a reprogrammable miniaturized micro-computer 200 with reconfigurable processing, sensor/actuator interfaces, and ultrasonic wireless interfaces may used as shown in FIG. 2. Primary elements of the ultrasonic wideband communication hardware node may include: (1) fully-programmable and re-configurable processing core; (2) ultrasonic interface amplification and processing/networking and are able to send and receive energy and data; and (3) power unit which allows the system to be recharged wirelessly using the Ultrasound signal. All the components may be built into a multilayer board layer circular board 210 as shown in FIG. 2.

The processing core unit includes (i) miniature ultra low-power field programmable gate array (FPGA) and (ii) a microcontroller unit (MCU). Their combination results in a “miniature computer” with hardware and software reconfigurability and relatively small packaging and low energy consumption. The miniaturized FPGA hosts the physical (PHY) layer communication functions and the MCU is in charge of data processing and of executing flexible and reconfigurable protocols for network, transport and application. In some embodiments, the FPGA and MCU can be replaced by a application specific circuit (ASICS) circuit to further reduce the size of the hardware and to reduce energy consumption.

The ultrasonic interface enables wireless connectivity and consists of a receiver (Rx) and a transmitter (Tx). The Rx includes a low-noise amplifier (LNA) and an analog-to-digital converter (ADC) to amplify and digital-convert received signals. The Tx embeds a digital-to-analog converter (DAC) and a power amplifier (PA) to analog-convert and amplify the digital waveform. The ultrasonic interface can use a piezoelectric crystal as emitter and or receiver, transceiver/transducer, of the ultrasound waves. In an embodiment a piezoelectric ceramic may be used as transceiver/transducer. Piezoelectric ceramics may have diverse shapes (plates, cylinders, hollow cylinders, spheres, semi-spheres) among others. Based on the geometry and design the piezo electric element may transmit unidirectionally, bi-directionally, multidirectional and or omni-directionally. Materials for construction of the piezo element may include crystal such as tourmaline, quartz, topaz, cane sugar, Rochelle salt, barium titanate, zirconate titanate, Langasite, Gallium orthophosphate, Lithium niobite, Lithium tantalate, Barium titanate, lead zirconate titanate (PZT), Potassium niobite, Sodium tungstate, Zinc oxide-Wurtzite structure, Sodium potassium niobite, Bismuth ferrite, Sodium niobite, Barium titanate, Bismuth titanate. Sodium bismuth titanate, and other ferro electrics. Ultrasonic piezos can also be created using bulk or nanostructured semiconductor crystal having non central symmetry. Piezoelectric can also be constructed from a polymer, Piezoelectric polymers (bulk polymers, voided charged polymers, and polymer composites). These materials may also be used to construct the piezo element in the form of a MEMs structure or a thin vibrating membrane.

In some embodiments, the ultrasonic transducer/transceiver is a component connected to the device/node electronics with the specific function of sending/receiving ultrasonic waves. In other embodiments the ultrasonic transceiver component can have other functions such as microphone, speaker, ultrasonic fingerprint scanner, watch bracelet, among others. In some embodiments a component of the utility device (e.g., phone, watch, bracelet, stereo, computer, payment station, etc.) is made from a piezo electric material, and thus may have a primary function associated with the overall function of the device itself, and a secondary function of ultrasonic communication. Such embodiments would include, casing, screens, ultrasonic energy harvesters, keyboards. The ultrasonic interface or complete ultrasonic communication hardware can be implemented in an adapter or interface device configured to connect to another electronic device and facilitate incorporation of ultrasonic communication and charging functionalities. Such adapters can include USB keys for identification purposes or hard keys for software authorization, adapter interface for RC jack, intelligent credit card interfaces for payment stations, intelligent building access cards, among other use cases. In further embodiments other wearables, implants and/or external devices can communicate in single node, bi-node and multi-node ultrasonic wideband networks.

The uses for a new communication technology that can connect multiple electronic devices has many uses in many fields. In terms of implantable devices, medical uses can include wireless pacemakers and defibrillators, neuro modulation devices, wireless neonatal care cribs, cochlear implants among others. Healthcare applications can also include an implantable chip that can serve as an identifier for the patients in order to access electronic health care records or to transmit healthcare information or reading from on body sensors. Intrabody networks can also be used to triage patients and send data securely from wearables or implants to the hospital network using a touchpad. There are several secure RF technologies that can send significant data such as Bluetooth LE, WIFI, among others, therefore intrabody networks are of special interest when high level of security is required and to avoid eavesdropping or in environment were these other forms of communication may not work, such as underwater, inside fluids, inside solids or in areas or high RF interference. Ultrasonic networks have many uses in underwater oil exploration and infrastructure monitoring, defense, aquaculture and scuba-diving among others, specific applications for wearables are associated mostly with identification, secure data transfer and payments among others. In an embodiment, the ultrasonic wideband technology is used to transfer ultrasonic data and or energy in order to communicate a wearable device to another wearable device or to an external device or entity. Although in air data transfer is possible using ultrasonic waves, the primary function would be to create an intrabody network that would constitute all or part of the communication channel to improve security and/or privacy of the network.

In an embodiment, a wearable transmitter, receiver and/or transceiver is placed in contact with any surface of the body, including but not limited to skin. The transceiver sends ultrasonic waves carrying data or energy through the skin and/or into other soft tissues, bone, cartilage, bodily fluids, organs and other tissue structure so that the signal reaches a different area of the body. In some embodiments, at the different location of the body a different wearable receiver or transceiver, collects the ultrasonic signal. In other embodiments touch between this location of the body and a receiver and/or transceiver on and external device will allow with communication to such a receiver. As described before, depending on the path of the signal through the body, a custom ultrasonic waveform and the specific piezoelectric transmitter with a known functional frequency may be required to optimize reliability and energy efficiency.

Many different wearables may be designed to include ultrasonic wideband communication hardware including watches, watch bracelets, bracelets, pads, necklaces, rings, earrings, clothing, shoes, arm-bands, backpacks, luggage, hats, glasses, earphones, virtual reality googles, headphones, helmets, hats and other headgear. External devices containing ultrasonic wideband electronics that may send receive ultrasonic wave/signals from the aforementioned wearables may include phones, computers, household electronics, payment stations, ATMs, mobiles computing devices, digital music players, building access pads, among others. In some applications a significant amount of external devices or infrastructure may already exist and replacing them for other with ultrasonic wideband capabilities, may not be practical. In such applications adapters and interfaces can be design as to add ultrasonic wideband communication capabilities to the existing device. Some embodiments of adapters or interfaces include USB or mini USB keys to interface with computers and other electronics that need ultrasonic wideband capabilities to communicate to a ultrasonic wideband enabled wearable. Other adapters include intelligent access and credit cards to interface with current access pad infrastructures in building or current paying stations. Further embodiments of Ultrasonic wideband adapters include wire dongles, RC jack keys, car key remotes or FOVs among others.

In some embodiments the data can be transmitted in a single direction or multi-directionally within intrabody networks, between two or more individuals with wearables in contract with their body. In such instances, two individuals with wearables communicate data using an ultrasonic based intrabody network as described above. In such an embodiment the intrabody signals of one or both of the individuals passes to the other by physical touch (e.g., handshake, first bump, skin to skin contact, etc.). In an example of this embodiment, the ultrasonic wave encoded with the data passes from the ultrasonic generator in the wearable into the first individuals body through the skin, and them progresses through the intrabody channel of such individual to a different section of their body, at which it passes to the second individual through direct contact into the second individuals skin and subsequent intrabody channel. The wave is then received and decoded by the wearable in contact with the skin of the second individual.

Due to the attributes of the intrabody communication technologies in terms of security, some of the primary applications include identification, access, and/or payments. Additional security may be attained by combining ultrasonic wideband intrabody communication with encryption, biometrics and/or passwords in order to attain multifactor authentication.

In an embodiment, bytes of data transferred using ultrasound wide band may also be encrypted using standard encryption techniques such as Triple data encryption standard, blowfish, Advanced Encryption Standard, Two-fish, symmetric and asymmetric encryption methods, Diffie-Hellman Key Exchange, ElGamal Encryption, Elliptic Curve Cryptography, honey encryption, and Quantum Key Distribution. The encrypted data from the transmitter is extracted at the receiver. In further embodiments of enhanced security the signal in the intrabody ultrasonic wideband network or other RF based sections of the communication path may be enhanced by including a secondary undetectable embedded signal within the first one with the protected data.

In an embodiment for authentication, passwords, biometrics, or other authentication data is stored within a wearable element in contact with the body. This data may then be transferred directly to an external device containing ultrasonic wideband electronics, or indirectly to the external device using a ultrasonic wideband adapter or interface. The communication channel will be kept within the body between the transmitter and the receiver. In such embodiments, unidirectional data will be transferred from the transmitter to the receiver, whereas in an embodiment bi-directional communication between the two nodes will be enabled. In some embodiments, part of the identification scheme will include biometric data, in such embodiments the biometric scanner may be located on the wearable node, the adapter node or the external device node independent if each node is the transmitter or receiver in the data transfer path. In some embodiments the biometric data is transferred from the biometric scanner (e.g., fingerprint reader, voice recognition microphone, camera for facial ID, eye iris scanner, etc.) located in the wearable or adapter to the external device, this external device then compares this received data to an internal or internet based biometrics database to ascertain the user identification. After a user is identified or otherwise authenticated, a user identifier can be corroborated and secure data may them be passed from the transmitter to the receiver, independent if the wearable is the transmitter or receiver of the secure data as authorized by the verified user. In other embodiments no further secured data needs be transferred, in such a case identification provides further access for the user in the system to execute further activities, in such scenarios this identification means may be used to replace a single or multiple passwords required for system access. In the application were user privacy is a concern, no biometric database is required. In such embodiments, biometric data is stored on the wearable, this data is them passed to the adapter or external device, wherever the biometric scanner is located. The scanner is used to retrieve biometric information from the end user (e.g., fingerprint, face image, voice, iris scan, etc.) and compared to that which was sent from the wearable to establish positive identification. After identification is established using this method, further secured data may be transferred through the intrabody network or any other data transfer means, or further access for the user to the system may be granted to complete other activities. This ensures that biometric data is passed only through the secure intrabody network and no information is required to be stored beyond that which is kept at the wearable. In an embodiment, the biometric data is stored within the wearable, and the external node or adaptor that holds the biometric scanner can scan the data and send it to the wearable device though the intrabody network. The data can be compared at the wearable device to identify the user and allow for further action between the nodes. Identification using the intrabody network could be used to access a house, office or any secured facility, rental of equipment, rental of means of transport, or other secured areas.

In a further use case for ultrasonic wideband technology as described herein, secured authorization and payment in accomplished by the means of an ultrasonic intrabody network. In an initial configuration of the system, a wearable device may store user payment information (e.g., passwords, credit card number, account numbers, pins, logins, security codes, etc.). Such information can be encoded and transferred through the arm wrist and hand or any other section of the body using an ultrasonic intrabody network. The information is then transferred through the skin to an external payment station. The payment station uses an ultrasonic receiver or transceiver to acquire the secured data and authorize the payment. In further embodiments, prior to sending the secure information a password is entered into the wearable to authorize the transfer of information. In other embodiments biometric data such as fingerprints, voice, iris or facial recognition is acquired by a scanner in the wearable and compared to biometric information stored also in the wearable to confirm user identification prior to sending the secure payment data through the intrabody network, after user identity is confirmed the secured data is authorized to by transferred by the system. In such an embodiment no database of biometric data is required in the external device. In other embodiments of use of the ultrasonic wideband technology the biometric scanner is located at the payment station. In such an embodiment the user biometric data may be sent from the wearable to the payment station in order to compare it to the data from the biometric scanner to authorize further transfer of information and payment (no external biometrical database required). In a further environment were the biometric scanner is located on the payment station, biometric data can be stared in the pay station or and external database for identification. In this further environment secured data from the wearable is only transferred to the pay station after the pay station sends back an authorization signal to the wearable after confirming user identity through biometrics. Both the authorization signal and secured data are transferred through the intrabody network.

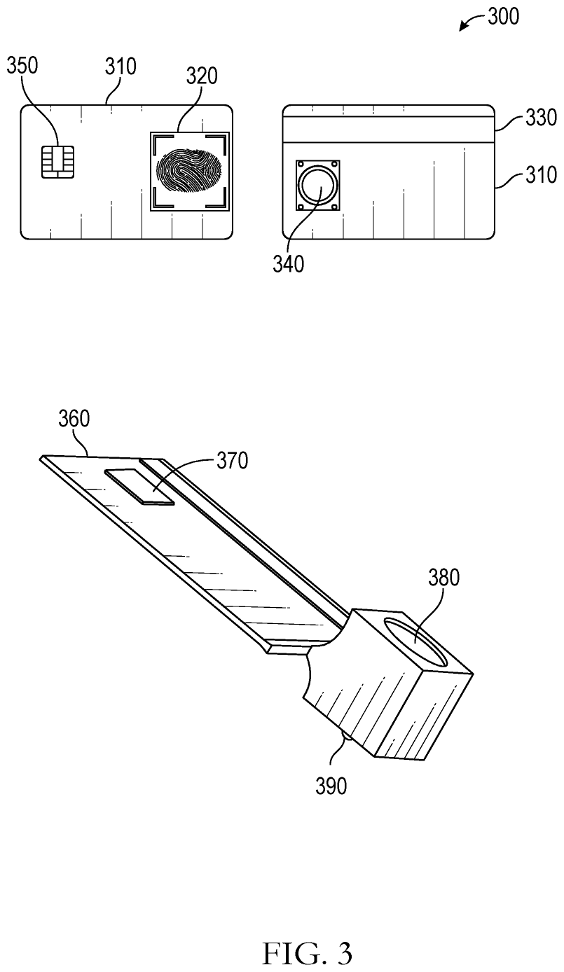

Referring to FIG. 3, an embodiment for uses of ultrasonic wideband technology for secure payment using an ultrasonic communication adapter is depicted. The illustrated embodiment 300 may enable the use of current payment stations worldwide without the need to add embedded ultrasonic communication hardware. As shown in FIG. 3, an adapter 310 has the form factor of a credit card to interface with current credit card payment stations. The adapter 310 has a credit card chip 350 and magnetic band 330 which do not hold user information but are only used as transfer means into the payment station of information received by the adapter 310. Therefore, if the adapter 310 is lost it is just a blank and poses no security risks. Because of their nature these adapters 310 may be then just be located at the stores or payment point and may not be needed to be carried by the user. In an embodiment, the adapter 310 has a biometric scanner 320, such as a fingerprint reader. The biometric scanner 320 may work by electrical, optical or ultrasonic means. In further embodiments of this adapter 310, the adapter 310 may also have an ultrasonic communication node 340 (e.g., electronics and piezoelectric components, etc.). Piezoelectric elements including the ultrasonic receiver or transceiver and electronics are described above to allow for ultrasonic wideband communication. In some embodiments, the adapter 310 has its own energy storage elements (e.g., batteries, capacitors, etc.) to power all the electronics in the adapter. These energy storage devices may be non rechargeable or rechargeable. Rechargeable embodiments may use cables or non-contact means including solar, and or induction, to replenish the energy in the batteries or other storage means. In further embodiments the energy may be recharged using ultrasound waves delivered through the intrabody network. In this configuration an ultrasonic wideband wearable can be used to initially send biometric data to the adapter through the intrabody network. The biometric data is then compared to that acquired by the biometric reader from the user and further action is authorized. If user if positively identified, credit card data stored in the wearable is sent to the adapter 310 and then into the payment station through the credit card chip or the magnetic strip. Some embodiments may require that the authorization to transfer data signal is sent back from the adapter to the wearable using two-way intrabody communication. In other embodiments both the biometric data and credit card data are initially transferred to the adapter from the wearable, but after user is identified is the payment data transferred into the magnet strip or chip to be sent to the payment station. In other examples of two-way communication, the biometric data from the scanner can be transferred from the biometric scanner in the adapter to the wearable, and compared within the wearable to positively identify the user.

In some embodiments of the adapter 310 in FIG. 3, the complete electronics are designed to fit within the normal volume of a credit card. In an embodiment to reduce costs and simplify fabrication, the adapter 310 would be configured such that a distal section 360 would have the width of a standard credit card and chip 370 to fit into the payment station, the elongated proximal section of the adapter may be thicker to fit the ultrasonic communication electronics 390 and piezo, energy storage and biometric scanner 380. In this embodiment, the proximal end containing these thicker elements would sit outside of the credit card slot in the payment station. In further embodiment, the distal and proximal section of the adapter may be detachable to simplify transport if carried by the end user.

In a different use case, covert payment transactions can be initiated by means of physical touch. More specifically, the smartwatch or other wearable can send—by means of ultrasonic waves confined in the human arm—a unique, pre-defined secret code to the smartphone or other receiver. The information can be transferred by physical touch exclusively, that is, there is no propagation of the information carrying signal outside of the body of the donor wearing the smartwatch. The secret code will be received by a background application running over the phone's operating system. After a given time interval, the app will establish a secure encrypted SSH connection with a remote server, and communicate the secret code to the server. Upon receipt and processing of the secret code, the remote server will execute a financial transaction by transferring a pre-defined sum of money between to bank account or two cryptocurrency accounts (the donor account, associated to the individual wearing the smartwatch, and the recipient account, associated to the owner of the smartwatch). Cryptocurrency transactions are unlinkable and untraceable as the algorithmic structure is based on ring signatures and stealth addresses to hide the identities of the sender and the receiver, as well as the amount of the transaction.

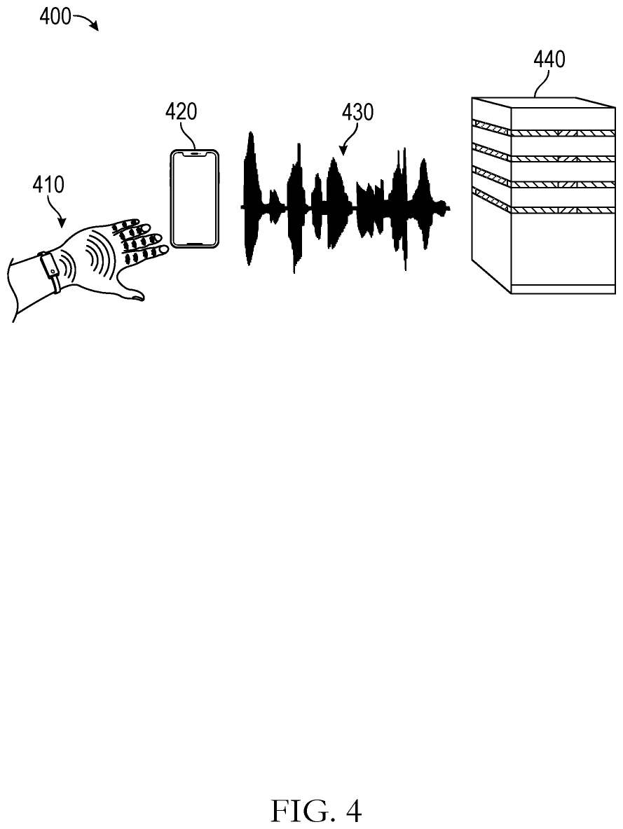

Referring to FIG. 4, a monetary transaction between an individual wearing a custom smart watch 410 (donor) and a mobile phone 420 (recipient) is depicted. By casually touching a mobile phone, and individual will be able to initiate a financial transaction through a phone running a background app. A secondary signal 430 to a remote processing server 440 may be undetectable as it may be embedded in a cellular communication. The payment, as well as the wireless signal carrying the signal initiating it, will be unperceivable to any entity listening to the wireless channel. For the application described above—transferring data to authorize a payment covertly and securely from a smartwatch to a smartphone by simply touching the smartphone—it may be desirable for the signal to propagate through the lower arm and the hand (without radiating outside the tissue), so as to make the data transfer undetectable to agents that are not in physical contact with the individual holding the smartwatch. Even in the scenario of physical contact between a third agent and the individual, the signal will not be detectable unless an ultrasound receiver is used in a specific time and location of the body, and the data carried by the signal may be further secured using advanced encryption. Further, on the receiver side the hardware can be implemented on the phone or receiver hardware itself or on a cover or accessory. For a cell phone or other similar electronic equipment, a case, screen or package may be constructed from a ultrasound carrying material or piezoelectric material, such that when touched it may receive the ultrasound signal and carry it to a specific site in the hardware were it can be further transported or processed. In other cases the embedded microphone in a typical electronic element such as a phone may be used to receive the signal directly by using a interface that can transmit through the gap of air between the human body (e.g., finger, etc.) and the surface of the microphone. Further, a resonance membrane can be used to transmit the signal from the body to the speaker. This membrane can be placed above and alternative site with field of view to the embedded microphone.

Although discussed with respect to certain embodiments, other embodiments may be implemented in many different wearables or implants allowing for many uses as deemed to be required in the field. In an extreme case, physical touch between two individuals with wearables or implants may allow for the same result with minimal alterations to the core technology.

As a result, certain embodiments of the disclosure may securely and reliably transfer data using ultrasonic wave transmitted through human tissue. Because the data may be sent through human tissue, the data may not be susceptible to theft or otherwise unauthorized access. Examples of data that can be transferred using such embodiments include authentication data, payment data, message data, and so forth.

One or more illustrative embodiments of the disclosure have been described above. The above-described embodiments are merely illustrative of the scope of this disclosure and are not intended to be limiting in any way. Accordingly, variations, modifications, and equivalents of embodiments disclosed herein are also within the scope of this disclosure. The above-described embodiments and additional and/or alternative embodiments of the disclosure will be described in detail hereinafter through reference to the accompanying drawings.

### Illustrative Processes and Use Cases

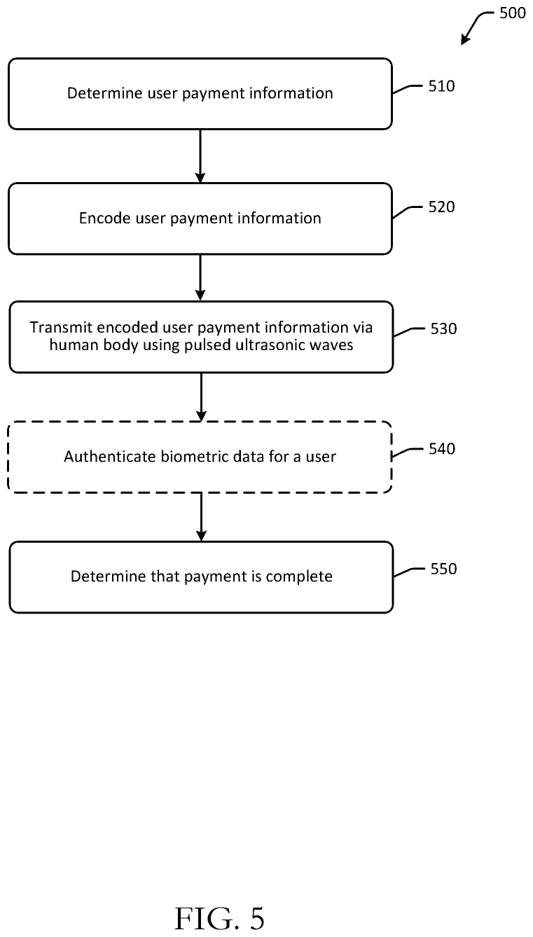

FIG. 5 is an example process flow diagram for payments using ultrasonic based communication schemes in accordance with one or more example embodiments of the disclosure. One or more operations or communications illustrated in FIG. 5 may occur concurrently or partially concurrently, while illustrated as discrete communications or operations for ease of illustration. One or more blocks of FIG. 5 may be optional and may be performed by a single device or across a distributed computing system.

At block 510 of the process flow 500, computer-executable instructions stored on a memory of a device, such as a wearable device, may be executed to determine user payment information. For example, a wearable device may determine user payment information for a user wearing the device. The wearable device may determine the user payment information by authenticating the user wearing the device, such as via a password, passcode, biometric screening, or other authentication. The user payment information may include one or more of credit card information, bank account information, user device identifier information, user address information, user account information, cryptocurrency information, and/or other payment information.

At block 520 of the process flow 500, computer-executable instructions stored on a memory of a device, such as a wearable device, may be executed to encode the user payment information. For example, a wearable device may encode the user payment information. The wearable device may encode the user payment information into one or more ultrasonic waves, such as a pulsed ultrasonic wave or a continuous ultrasonic wave.

At block 530 of the process flow 500, computer-executable instructions stored on a memory of a device, such as a wearable device, may be executed to transmit the encoded user payment information via a human body using pulsed ultrasonic waves. For example, a wearable device may transmit the encoded user payment information via a human body using pulsed ultrasonic waves. The wearable device may transmit the encoded user payment information via a human body using pulsed ultrasonic waves or continuous ultrasonic waves, such that the waves propagate through the human or animal tissue. The encoded user payment information may be received by a receiver that may be internal or external relative to the human body. In some embodiments, the encoded user payment information may be at a payment terminal, such as a credit card terminal.

At optional block 540 of the process flow 500, computer-executable instructions stored on a memory of a device, such as a wearable device or a payment terminal, may be executed to authenticate biometric data for a user. For example, a wearable device or a payment terminal may authenticate the user associated with the user payment data using biometric data, which may include fingerprint data, iris data, or other biometric information.

At block 550 of the process flow 500, computer-executable instructions stored on a memory of a device, such as a wearable device, may be executed to determine that payment is complete. For example, a wearable device may determine that payment is complete based at least in part on an acknowledgment communication received from an external device. For example, a payment terminal may receive the user payment information, and may authenticate the user, and then may send an acknowledgment message or confirmation that may be received by the wearable device to confirm completion of the payment. The wearable device may therefore determine that the payment is complete.

In one embodiment, an ultrasonic intrabody wireless communication system may include a wearable device in contact with a surface of a human body, the wearable device comprising an ultrasonic wave generator configured to transmit pulsed ultrasonic waves, and an external receiver configured to receive the pulsed ultrasonic waves and determine data encoded in the pulsed ultrasonic waves. The pulsed ultrasonic waves encode a single bit of data in multiple pulses through a pseudorandom time hopping scheme. Some embodiments may include an adapter configured to communicate with a device that does not have ultrasonic communication capabilities. At least part of the data transmitted through the intrabody network may include biometric information. Such biometric data may include user information associated with one or more of voice data, fingerprint data, or retinal data. The pulsed ultrasonic waves may be transmitted through a human hand.

In another embodiment, an ultrasonic intrabody wireless communication system for secure access or payment may include a wearable device in contact with a surface of a human body, the wearable device including an ultrasonic wave transceiver that is configured to communicate using ultrasonic waves, an external transceiver configured to decode data encoded within the ultrasonic waves, and a biometric scanner configured to identify a biometric feature associated with the human body. Communication between the wearable device and external transceiver is at least partially transferred through an ultrasonic intrabody network. The wearable device may be configured to communicate with the external transceiver using pulsed ultrasonic waves. Data associated with the biometric feature may be stored only at the wearable device. Data associated with the biometric feature may be at least partially transmitted through an intrabody network. The system may be configured to transmit intrabody data through a human hand. The external transceiver can be a credit card payment station or a secure access control lock. The system can use pulse ultrasound signals for communication.

In another embodiment, a system for ultrasonic intrabody communication may include a transmitter configured to send data encoded in ultrasonic waves at least partially through an intrabody communications channel, and an adapter configured to receive data encoded in the ultrasonic waves, where the adapter comprises an interface within the adapter that is configured to communicate with an external device that has no direct ultrasonic data connectivity. For example, the external device may not be configured to receive, decode, or otherwise determine ultrasonic data connectivity. The adapter may be configured to communicate with a payment station or automated teller machine. The adapter may be configured to communicate with a secure access interface. The transmitter may be disposed within a wearable device in contact with a human body. The interface may be a magnetic strip or a credit card payment chip. The system may include at least one biometric scanner. The at least one biometric scanner may be disposed on the adapter. The adapter may be used for a multifactor authentication system.

One or more operations of the process flows or use cases of FIGS. 1-5 may have been described above as being performed by a user device, or more specifically, by one or more program modules, applications, or the like executing on a device. It should be appreciated, however, that any of the operations of process flows or use cases of FIGS. 1-5 may be performed, at least in part, in a distributed manner by one or more other devices, or more specifically, by one or more program modules, applications, or the like executing on such devices. In addition, it should be appreciated that processing performed in response to execution of computer-executable instructions provided as part of an application, program module, or the like may be interchangeably described herein as being performed by the application or the program module itself or by a device on which the application, program module, or the like is executing. While the operations of the process flows or use cases of FIGS. 1-5 may be described in the context of the illustrative remote server, it should be appreciated that such operations may be implemented in connection with numerous other device configurations.

The operations described and depicted in the illustrative process flows or use cases of FIGS. 1-5 may be carried out or performed in any suitable order as desired in various example embodiments of the disclosure. Additionally, in certain example embodiments, at least a portion of the operations may be carried out in parallel. Furthermore, in certain example embodiments, less, more, or different operations than those depicted in FIGS. 1-5 may be performed.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

### Illustrative Device Architecture

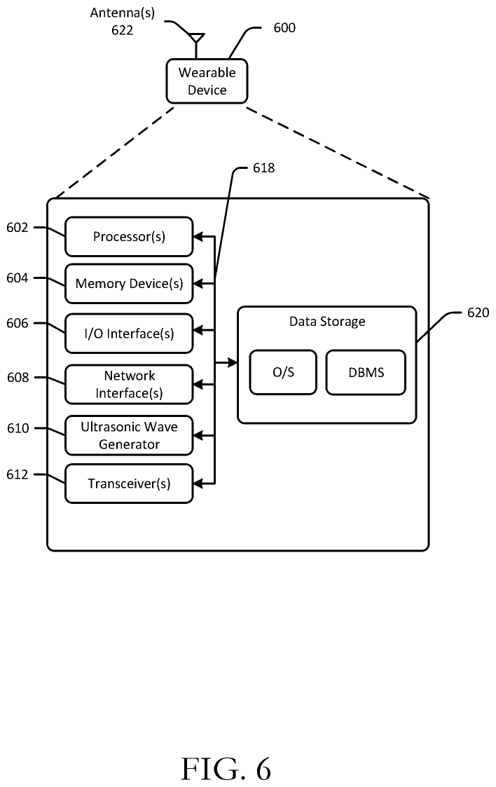

FIG. 6 is a schematic illustration of example computer architecture of an illustrative wearable device 600 in accordance with one or more example embodiments of the disclosure. The wearable device 600 may include any suitable computing device capable of receiving and/or generating data including, but not limited to, a watch, a wristband, or the like. The wearable device 600 may correspond to an illustrative device configuration for the devices of FIGS. 1-5.

The wearable device 600 may be configured to communicate via one or more devices using ultrasonic waves and/or via networks with one or more servers, user devices, or the like. Example network(s) may include, but are not limited to, any one or more different types of communications networks such as, for example, cable networks, public networks (e.g., the Internet), private networks (e.g., frame-relay networks), wireless networks, cellular networks, telephone networks (e.g., a public switched telephone network), or any other suitable private or public packet-switched or circuit-switched networks. Further, such network(s) may have any suitable communication range associated therewith and may include, for example, global networks (e.g., the Internet), metropolitan area networks (MANs), wide area networks (WANs), local area networks (LANs), or personal area networks (PANs). In addition, such network(s) may include communication links and associated networking devices (e.g., link-layer switches, routers, etc.) for transmitting network traffic over any suitable type of medium including, but not limited to, coaxial cable, twisted-pair wire (e.g., twisted-pair copper wire), optical fiber, a hybrid fiber-coaxial (HFC) medium, a microwave medium, a radio frequency communication medium, a satellite communication medium, or any combination thereof.

In an illustrative configuration, the wearable device 600 may include one or more processors (processor(s)) 602, one or more memory devices 604 (generically referred to herein as memory 604), one or more input/output (I/O) interface(s) 606, one or more network interface(s) 608, one or more ultrasonic wave generator(s) 610, one or more transceivers 612, and data storage 620. The wearable device 600 may further include one or more buses 618 that functionally couple various components of the wearable device 600. The wearable device 600 may further include one or more antenna(s) 622 that may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals, a Near Field Communication (NFC) antenna for transmitting or receiving NFC signals, and so forth. These various components will be described in more detail hereinafter.

The bus(es) 618 may include at least one of a system bus, a memory bus, an address bus, or a message bus, and may permit exchange of information (e.g., data (including computer-executable code), signaling, etc.) between various components of the wearable device 600. The bus(es) 618 may include, without limitation, a memory bus or a memory controller, a peripheral bus, an accelerated graphics port, and so forth. The bus(es) 618 may be associated with any suitable bus architecture including, without limitation, an Industry Standard Architecture (ISA), a Micro Channel Architecture (MCA), an Enhanced ISA (EISA), a Video Electronics Standards Association (VESA) architecture, an Accelerated Graphics Port (AGP) architecture, a Peripheral Component Interconnects (PCI) architecture, a PCI-Express architecture, a Personal Computer Memory Card International Association (PCMCIA) architecture, a Universal Serial Bus (USB) architecture, and so forth.

The memory 604 of the wearable device 600 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In certain example embodiments, volatile memory may enable faster read/write access than non-volatile memory. However, in certain other example embodiments, certain types of non-volatile memory (e.g., FRAM) may enable faster read/write access than certain types of volatile memory.

The data storage 620 may include removable storage and/or non-removable storage including, but not limited to, magnetic storage, optical disk storage, and/or tape storage. The data storage 620 may provide non-volatile storage of computer-executable instructions and other data. The memory 604 and the data storage 620, removable and/or non-removable, are examples of computer-readable storage media (CRSM) as that term is used herein.

The data storage 620 may store computer-executable code, instructions, or the like that may be loadable into the memory 604 and executable by the processor(s) 602 to cause the processor(s) 602 to perform or initiate various operations. The data storage 620 may additionally store data that may be copied to memory 604 for use by the processor(s) 602 during the execution of the computer-executable instructions. Moreover, output data generated as a result of execution of the computer-executable instructions by the processor(s) 602 may be stored initially in memory 604, and may ultimately be copied to data storage 620 for non-volatile storage.

More specifically, the data storage 620 may store one or more operating systems (O/S) 622; one or more database management systems (DBMS) 624; and one or more program module(s), applications, engines, computer-executable code, scripts, or the like. Some or all of these module(s) may be sub-module(s). Any of the components depicted as being stored in data storage 620 may include any combination of software, firmware, and/or hardware. The software and/or firmware may include computer-executable code, instructions, or the like that may be loaded into the memory 604 for execution by one or more of the processor(s) 602. Any of the components depicted as being stored in data storage 620 may support functionality described in reference to correspondingly named components earlier in this disclosure.

The data storage 620 may further store various types of data utilized by components of the wearable device 600. Any data stored in the data storage 620 may be loaded into the memory 604 for use by the processor(s) 602 in executing computer-executable code. In addition, any data depicted as being stored in the data storage 620 may potentially be stored in one or more datastore(s) and may be accessed via the DBMS 624 and loaded in the memory 604 for use by the processor(s) 602 in executing computer-executable code. The datastore(s) may include, but are not limited to, databases (e.g., relational, object-oriented, etc.), file systems, flat files, distributed datastores in which data is stored on more than one node of a computer network, peer-to-peer network datastores, or the like.

The processor(s) 602 may be configured to access the memory 604 and execute computer-executable instructions loaded therein. For example, the processor(s) 602 may be configured to execute computer-executable instructions of the various program module(s), applications, engines, or the like of the wearable device 600 to cause or facilitate various operations to be performed in accordance with one or more embodiments of the disclosure. The processor(s) 602 may include any suitable processing unit capable of accepting data as input, processing the input data in accordance with stored computer-executable instructions, and generating output data. The processor(s) 602 may include any type of suitable processing unit including, but not limited to, a central processing unit, a microprocessor, a Reduced Instruction Set Computer (RISC) microprocessor, a Complex Instruction Set Computer (CISC) microprocessor, a microcontroller, an Application Specific Integrated Circuit (ASIC), a Field-Programmable Gate Array (FPGA), a System-on-a-Chip (SoC), a digital signal processor (DSP), and so forth. Further, the processor(s) 602 may have any suitable microarchitecture design that includes any number of constituent components such as, for example, registers, multiplexers, arithmetic logic units, cache controllers for controlling read/write operations to cache memory, branch predictors, or the like. The microarchitecture design of the processor(s) 602 may be capable of supporting any of a variety of instruction sets.

Referring now to other illustrative components depicted as being stored in the data storage 620, the O/S 622 may be loaded from the data storage 620 into the memory 604 and may provide an interface between other application software executing on the wearable device 600 and hardware resources of the wearable device 600. More specifically, the O/S 622 may include a set of computer-executable instructions for managing hardware resources of the wearable device 600 and for providing common services to other application programs (e.g., managing memory allocation among various application programs). The O/S 622 may include any operating system now known or which may be developed in the future including, but not limited to, any server operating system, any mainframe operating system, or any other proprietary or non-proprietary operating system.

The DBMS 624 may be loaded into the memory 604 and may support functionality for accessing, retrieving, storing, and/or manipulating data stored in the memory 604 and/or data stored in the data storage 620. The DBMS 624 may use any of a variety of database models (e.g., relational model, object model, etc.) and may support any of a variety of query languages. The DBMS 624 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like. In those example embodiments in which the wearable device 600 is a mobile device, the DBMS 624 may be any suitable light-weight DBMS optimized for performance on a mobile device.

Referring now to other illustrative components of the wearable device 600, the input/output (I/O) interface(s) 606 may facilitate the receipt of input information by the wearable device 600 from one or more I/O devices as well as the output of information from the wearable device 600 to the one or more I/O devices. The I/O interface(s) 606 may also include a connection to one or more of the antenna(s) 622 to connect to one or more networks via a wireless local area network (WLAN) (such as Wi-Fi) radio, Bluetooth, ZigBee, and/or a wireless network radio, such as a radio capable of communication with a wireless communication network such as a Long Term Evolution (LTE) network, WiMAX network, 3G network, ZigBee network, etc.

The wearable device 600 may further include one or more network interface(s) 608 via which the wearable device 600 may communicate with any of a variety of other systems, platforms, networks, devices, and so forth. The network interface(s) 608 may enable communication, for example, with one or more wireless routers, one or more host servers, one or more web servers, and the like via one or more of networks.

The antenna(s) 622 may additionally, or alternatively, include a Wi-Fi antenna configured to transmit or receive signals in accordance with established standards and protocols, such as the IEEE 802.11 family of standards, including via 2.4 GHz channels (e.g., 802.11b, 802.11g, 802.11n), 5 GHz channels (e.g., 802.11n, 802.11ac), or 60 GHz channels (e.g., 802.11ad). In alternative example embodiments, the antenna(s) 622 may be configured to transmit or receive radio frequency signals within any suitable frequency range forming part of the unlicensed portion of the radio spectrum.

The antenna(s) 622 may additionally, or alternatively, include a GNSS antenna configured to receive GNSS signals from three or more GNSS satellites carrying time-position information to triangulate a position therefrom. Such a GNSS antenna may be configured to receive GNSS signals from any current or planned GNSS such as, for example, the Global Positioning System (GPS), the GLONASS System, the Compass Navigation System, the Galileo System, or the Indian Regional Navigational System.

The transceiver(s) 612 may include any suitable radio component(s) for—in cooperation with the antenna(s) 622—transmitting or receiving radio frequency (RF) signals and/or ultrasonic wave signals in the bandwidth and/or channels corresponding to the communications protocols utilized by the wearable device 600 to communicate with other devices. The transceiver(s) 612 may include hardware, software, and/or firmware for modulating, transmitting, or receiving—potentially in cooperation with any of antenna(s) 622—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. The transceiver(s) 612 may further include hardware, firmware, or software for receiving GNSS signals. The transceiver(s) 612 may include any known receiver and baseband suitable for communicating via the communications protocols utilized by the wearable device 600. The transceiver(s) 612 may further include a low noise amplifier (LNA), additional signal amplifiers, an analog-to-digital (A/D) converter, one or more buffers, a digital baseband, or the like.

The ultrasonic wave generator(s) 610 may include or may be capable of generating ultrasonic waves, such as pulsed ultrasonic waves, with data encoded therein.

It should further be appreciated that the wearable device 600 may include alternate and/or additional hardware, software, or firmware components beyond those described or depicted without departing from the scope of the disclosure. More particularly, it should be appreciated that software, firmware, or hardware components depicted as forming part of the wearable device 600 are merely illustrative and that some components may not be present or additional components may be provided in various embodiments. While various illustrative program module(s) have been depicted and described as software module(s) stored in data storage 620, it should be appreciated that functionality described as being supported by the program module(s) may be enabled by any combination of hardware, software, and/or firmware. It should further be appreciated that each of the above-mentioned module(s) may, in various embodiments, represent a logical partitioning of supported functionality. This logical partitioning is depicted for ease of explanation of the functionality and may not be representative of the structure of software, hardware, and/or firmware for implementing the functionality. Accordingly, it should be appreciated that functionality described as being provided by a particular module may, in various embodiments, be provided at least in part by one or more other module(s). Further, one or more depicted module(s) may not be present in certain embodiments, while in other embodiments, additional module(s) not depicted may be present and may support at least a portion of the described functionality and/or additional functionality. Moreover, while certain module(s) may be depicted and described as sub-module(s) of another module, in certain embodiments, such module(s) may be provided as independent module(s) or as sub-module(s) of other module(s).

Program module(s), applications, or the like disclosed herein may include one or more software components including, for example, software objects, methods, data structures, or the like. Each such software component may include computer-executable instructions that, responsive to execution, cause at least a portion of the functionality described herein (e.g., one or more operations of the illustrative methods described herein) to be performed.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

That which is claimed is:

1. An ultrasonic intrabody wireless communication system comprising:
a wearable device in contact with a surface of a human body, the wearable device comprising an ultrasonic wave generator configured to transmit ultrasonic waves; and
an external receiver configured to receive the ultrasonic waves and determine data encoded in the ultrasonic waves.

2. The ultrasonic intrabody wireless communication system of claim 1, wherein the ultrasonic waves are pulsed ultrasonic waves that encode each bit of data in one or more pulses.

3. The ultrasonic intrabody wireless communication system of claim 1, further comprising:
an adapter configured to communicate with a device that does not have ultrasonic communication capabilities.

4. The ultrasonic intrabody wireless communication system of claim 1, wherein at least part of the data transmitted through the intrabody network comprises authentication information.

5. The ultrasonic intrabody wireless communication system of claim 1, wherein at least part of the data transmitted through the intrabody network comprises biometric data that includes user information associated with one or more of voice data, fingerprint data, electrocardiogram (ECG) data, electro-encephalogram (EEG) data, or retinal data.

6. An ultrasonic intrabody wireless communication system for secure access or payment comprising:
a wearable device in contact with a surface of a human body, the wearable device comprising an ultrasonic wave transceiver that is configured to communicate using ultrasonic waves;
an external transceiver configured to decode data encoded within the ultrasonic waves; and
a biometric scanner configured to identify a biometric feature associated with the human body;
wherein communication between the wearable device and external transceiver is at least partially transferred through an ultrasonic intrabody network.

7. The ultrasonic intrabody wireless communication system of claim 6, wherein the wearable device is configured to communicate with the external transceiver using pulsed ultrasonic waves.

8. The ultrasonic intrabody wireless communication system of claim 6, wherein data associated with the biometric feature is stored only at the wearable device.

9. The ultrasonic intrabody wireless communication system of claim 6, wherein data associated with the biometric feature is at least partially transmitted through an intrabody network.

10. The ultrasonic intrabody wireless communication system of claim 6, wherein the system is configured to transmit intrabody data through a human hand.

11. The ultrasonic intrabody wireless communication system of claim 6, wherein the external transceiver is a credit card payment station or a secure access control lock.

12. The ultrasonic intrabody wireless communication system of claim 6, wherein the system uses pulse ultrasound signals for communication.

13. A system for ultrasonic intrabody communication comprising:
a transmitter configured to send data encoded in ultrasonic waves at least partially through an intrabody communications channel; and
an adapter configured to receive data encoded in the ultrasonic waves, wherein the adapter comprises an interface within the adapter that is configured to communicate with an external device that has no direct ultrasonic data connectivity.

14. The system of claim 13, wherein the adapter is configured to communicate with a payment station or automated teller machine.

15. The system of claim 13, wherein the adapter is configured to communicate with a secure access interface.

16. The system of claim 13, wherein the transmitter is disposed within a wearable device in contact with a human body.

17. The system of claim 13, wherein the interface is a magnetic strip or a credit card payment chip.

18. The system of claim 13, further comprising:
at least one biometric scanner disposed on the adapter.

19. The system of claim 13, wherein the adapter is used for a multifactor authentication system.

20. A method comprising:
determining, by a wearable device comprising one or more computer processors coupled to memory, payment data to communicate via ultrasonic waves transmitted through human tissue;
determining the ultrasonic waves, wherein the payment data is encoded in the ultrasonic waves;
generating the ultrasonic waves using an ultrasonic wave generator; and
determining that the payment data is received by an external device;
wherein the ultrasonic waves are confined to the human tissue and can be received only via physical contact.
